(19) United States
(12) Patent Application Publication
(10) Pub. No.: US 2004/0116854 A1
(43) Pub. Date: Jun. 17, 2004

(54) Syringe retractable needle and method

(76) Inventors: Ramzi F. Abulhaj, Miramar, FL (US); Xian Y. Li, Tai Zhou City (CN); Jack E. Dominik, Miami Lakes, FL (US)

Correspondence Address: H. JAY SPIEGEL & ASSOCIATES, P.O. BOX 444, MOUNT VERNON, VA, 22121, US

(21) Appl. No.: 10/317,515

(22) Filed: Dec. 12, 2002

(51) Int. Cl. A61M 5/00

(52) U.S. Cl. 604/110; 604/187

(57) ABSTRACT

A syringe carrying a needle with a barrel and hollow piston for dispersing an injectant through the end of the retractable needle. A resilient chuck seal is independently formed for positioning at the transition from the syringe barrel to the needle shank holding portion of the end of the barrel. The needle is propelled by the coaxial spring into the interior portion of the piston and totally retracted when the chuck seal is released. The method of the present invention relates to the use of a symmetrical chuck seal, annular in configuration, for not only gripping the head of the retractable needle, but simultaneously serving as the seal for the needle at the lower portion of the barrel so that the injectant escapes through the hollow syringe needle.

### FIELD OF THE INVENTION

This Application relates to syringes. More specifically, it relates to syringes in which the needle retracts after the injection has been completed. These syringes are basically found in U.S. Class 604, in various sub-classes, and in International Class A/61, also with various sub-classes.

### BACKGROUND OF THE INVENTION

The quest for a successfully operable syringe with a retractable needle is not new. Efforts were made as early as 1956 in U.S. Pat. No. 2,752,918, issued to J. W. H. Uytenbogaart. A subsequent effort was made by Saenz in U.S. Pat. No. 3,046,985. A more recent attempt at a commercial embodiment is disclosed in U.S. Pat. No. 6,015,438 issued to Thomas J. Shaw. This Patent has been assigned to Retractable Technologies, Inc. of Little Elm, Tex. Numerous Shaw patents exist relating to syringes with retractable needles, including but not limited to U.S. Pat. Nos. 5,267,961; 5,385,551; 5,389,076; 5,423,758; 5,578,011; 5,632,733; 5,637,092; 5,632,733; 5,817,058; 5,989,220; 5,997,512; 6,090,077; 6,210,371; 6,221,055; and 6,379,337. The Shaw patent 6,015,438 introduces two problems into the injection as well as retractable phases of use. The first is the force required to move the independent ring out of its position where it holds the needle head so that the needle head can be released to retract. The second problem relates to moving the seal at the end of the plunger so that the plunger end can engage the independent ring to dislodge it and thereby permit the needle head to retract based upon being spring loaded with a coaxially mounted coil spring. A further problem is introduced in as much as careful assembly must be made to place the seal in the right position at the end of the plunger since it is in a position to slide along the plunger. Also, the ring which holds the head of the needle is a separate part and it requires proper orientation and insertion around the head of the needle so that the force applied for injections is sufficient to hold the needle in place during the injection process and yet orient the ring so that it can be dislodged by the end of the plunger. The forces involved in dislodging the needle quite often are difficult to accomplish. Many elderly and arthritic persons with diabetes and other ailments requiring injectants find these forces, prior to retraction, difficult to perform. Furthermore, there is a general apprehension when utilizing any syringe if it becomes difficult to press the plunger forward.

Yet another attempt at a retractable syringe is revealed in U.S. Pat. No. 5,019,044 issued to Tsao in which a pair of opposed tongs hold the needle head in place and are released at the end of the injection stroke. Such a construction, particularly because it requires a threaded engagement of the holder of the tongs, is inherently expensive to not only make the piece parts, but assemble them as well. In addition, there is no effective seal at the head of the needle in the nature of what an elastomeric seal will deliver.

In the environment as described, what is needed is a syringe with a retractable needle which eliminates the utilization of sliding members to hold the needle in place, and thereafter dislodge it. Moreover, what is required is a piston end barrel in which the end seal is permanently mounted so that it cannot slide and induce the possibility of leakage. Unquestionably it is desirable to have a seal at the needle head which, in addition to sealing, accomplishes the holding and release of the needle assembly.

The present invention is directed to dealing with the many deficiencies of the prior art just described.

### SUMMARY OF THE INVENTION

The present invention is a syringe carrying a needle assembly coupled to a barrel and piston for dispersing an injectant through the end of the needle in which the needle is spring loaded to retract after injection is completed. A resilient chuck seal is independently formed for positioning at the transition from the syringe barrel to the needle shank holding portion of the end of the barrel. The chuck seal is annular in configuration with a chamfer face tapering toward the inner smaller diameter of the chuck seal. When in place the chuck seal is secured to the head of the needle, while the needle is spring loaded to retract into the barrel, but held in place by the resilient chuck seal. At the end of the piston there is a removable piston plug. Such plug prevents the injectant from leaking into the interior of the hollow piston. The piston end has an annular chuck seal key proportioned to engage the chamfered face of the of the chuck seal after the piston plug has been dislodged and moved interiorly of the piston barrel. Then the key, upon engaging the chamfer, causes the holding end of the chuck seal to release the head of the needle. The needle is thereafter propelled by the needle coaxial spring into the interior portion of the piston and totally retracted. Provision is made for an interlock between the piston cap and the interior portion of the end of the cap end of the barrel. The method of the present invention relates to the use of a chuck seal, annular in configuration, for not only gripping the head of the retractable needle, but simultaneously serving as the seal for the needle at the lower portion of the barrel so that the injectant can only escape through the hollow syringe needle. A further feature of the invention is the provision of an annular key on the end of the to open the chuck seal at the end of the injection stroke thereby releasing the compression grip on the head of the syringe needle. Thereafter the needle is propelled by its coaxially mounted spring into the hollow interior portion of the piston. Immediately prior to the release of the needle, the piston and the plug, which keep the interior portion of the piston essentially dry, are dislodged. A seal surrounds the end of the piston which is fixedly secured in a seal groove at the end of the piston so that the piston seal is immovable along the piston and also the chuck seal is linearly immovable at the end of the stroke. This results in a condition immediately prior to release of the needle where a very precise amount of pressure is felt by the user which, when overcome, not only removes the plug at the end of the piston but opens up the chuck seal. When the chuck seal opens it permits the needle to be propelled into the hollow portion of the piston into a position of safety. At the same time means are provided at the end of the barrel to coact and secure the piston cap at the end of the barrel which is pushed by the thumb of the attendant.

One chuck seal has slots at 120° spacing at one end and three slots at 120° spacing at the other end, with the spacings on each end rotated from the other so that there is one slot at every 60° circumferentially around the chuck seal.

In an alternative embodiment the chuck seal is substantially identical with the first embodiment as to the engagement by the key and the provision for a chamfered face. Instead of the three slots defining three separate jaws on different spacings, an interior annular cut proceeds from the face opposite the chamfered face to an inner portion thereby defining a chamber which cause the exterior skirt of the chuck seal to be cantileverly supported at the end close to the chamfer to thereby make removal of the needle head easier when the chuck key contacts the chamfered face of the chuck seal jaws.

In view of the foregoing it is a principal object of the present invention to provide a syringe retractable needle and method which reduce the parts involved to an irreducible minimum, while at the same time providing for dislodgement of the retractable needle from its operative position by single firm but not difficult amount of pressure from the user.

A further object of one aspect of the present invention is to provide critical parts including the chuck seal for the needle and the seal for the which are symmetrical and hence non-directional when positioned in place, not requiring any specific orientation in order to be operable.

Yet another object of the present invention relates to lockingly engaging the retractable needle into position, after it has been retracted, to inhibit the reuse of the syringe and therefore dictate that it must be disposed of after a single use operation.

A further aspect of the invention relates to simplifying the filling of the syringe with a retractable needle by providing adequate relief ports and a single non-sliding seal at the end of the so that a uniform withdrawal of the injectant from a vial, into which a needle has been inserted, can be achieved.

### DESCRIPTION OF ILLUSTRATIVE EMBODIMENTS

Further objects and advantages of the present invention will become apparent as the following description of illustrative embodiments and methods are described, taken in conjunction with the accompanying illustrative drawings in which:

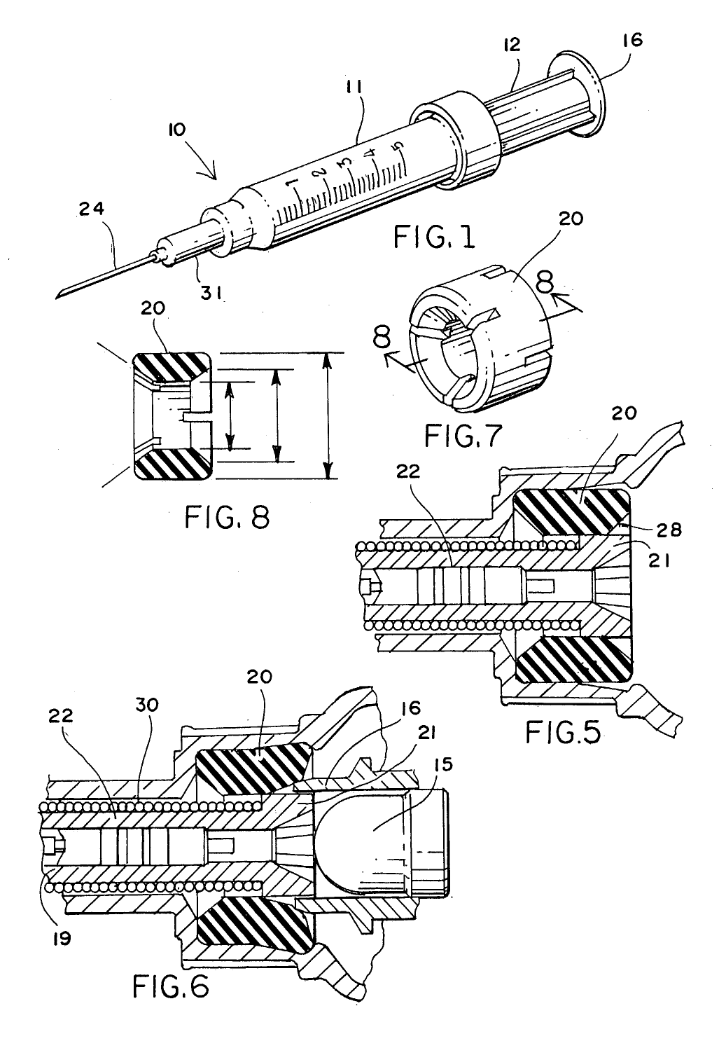

FIG. 1 is a perspective view of the subject illustrative syringe retractable needle and method showing the principal parts;

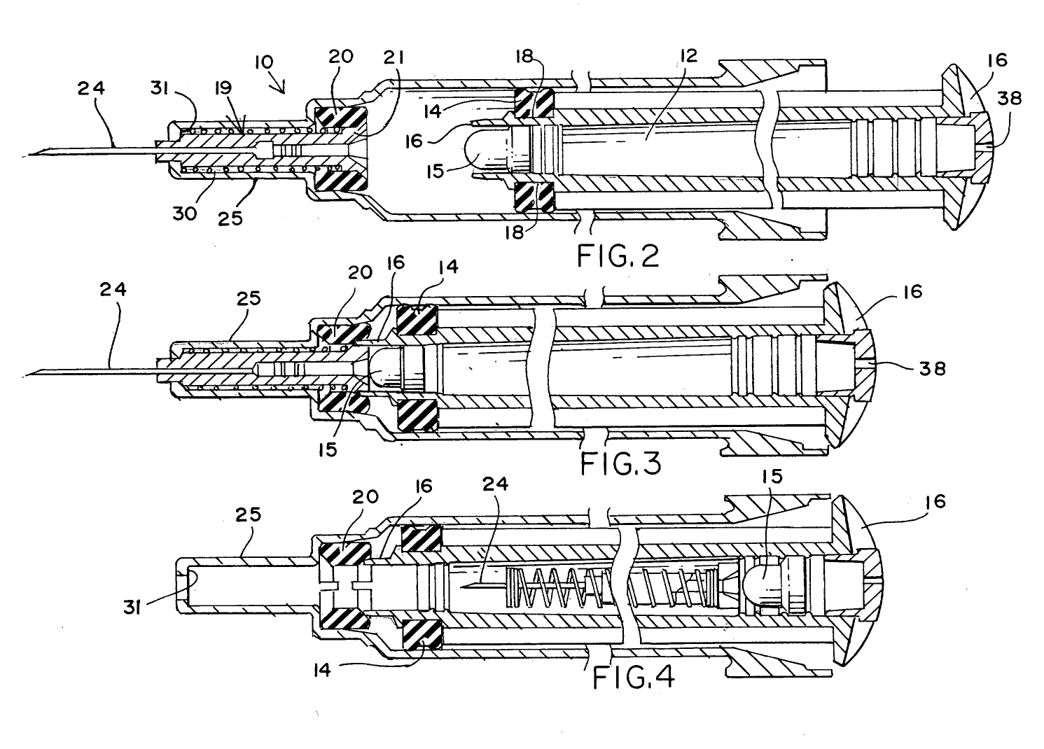

FIG. 2 is a longitudinal section taken along from one end to the other of the syringe retractable needle shown in FIG. 1 and illustrating the needle in the position of either filling the syringe barrel by withdrawal, or half injecting the contents of the syringe after the barrel has been filled to the prescribed amount of injectant and the user begins moving the forward;

FIG. 3 is a longitudinal section of FIG. 1 taken on the same scale as FIG. 2 but illustrating the piston at a position just immediately prior to the end of the stroke of the piston and opening the jaws of the chuck seal to release the head of the needle and at the same time dislodge the piston plug to retract along with the needle;

FIG. 4 is a view taken in the same scale, along the same section line as FIGS. 2 and 3, showing the needle fully retracted and the head of the immediately prior to securing itself interiorly of the piston cap end of the barrel;

FIG. 5 is an enlarged partially broken view of the first embodiment chuck seal as it is in position to seal the needle at the end of the barrel and at the same time secure the needle head in place by annular resilient contact with the needle head;

FIG. 6 is a view in the same scale and same section as FIG. 5 but showing the end of the barrel, and more specifically the barrel key, which is an annular ring at the end of the barrel engaging the chamfered face of the chuck seal immediately prior to the release of the needle head which in turn will cause the plug at the end of the piston to continually retract interiorly of the piston as the coil spring, which coaxially surrounds the needle shank and abuts against the underneath portion of the needle head, propels the needle into the interior portion of the barrel;

FIG. 7 is an enlarged perspective view of the chuck seal showing three slots on each side of the chuck seal of the first embodiment which are spaced at 120° apart from each other on each end of the chuck seal, but in offset relationship so that going from front to rear there is a relief slot in everyone of six slots at a spacing of 60°;

FIG. 8 is a cutaway sectional view of the first embodiment chuck seal taken at section 8-8 of FIG. 7;

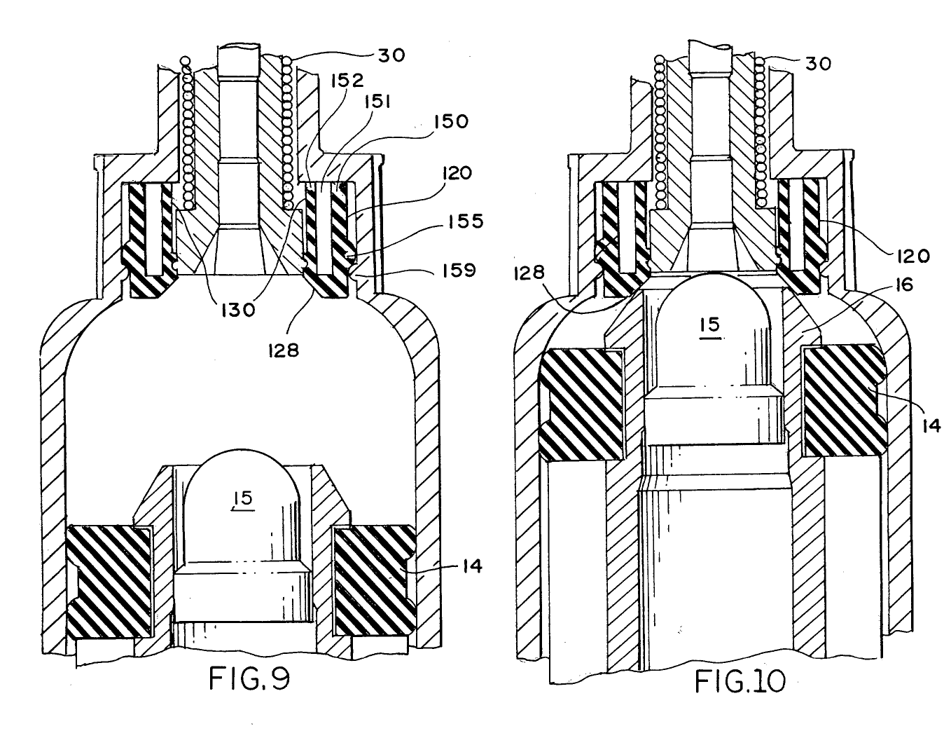

FIG. 9 is a partially broken section view of the alternative embodiment chuck seal showing the piston at a position remote from the chuck seal;

FIG. 10 is a sequential view in operation of the alternative embodiment chuck seal showing the chuck ring immediately after contact with the alternative embodiment chuck seal;

FIG. 11 is a sequential view from that of FIG. 10 showing the chuck ring penetrating the alternative chuck seal, partially crushing the same, simultaneously releasing the needle head and the piston plug; and

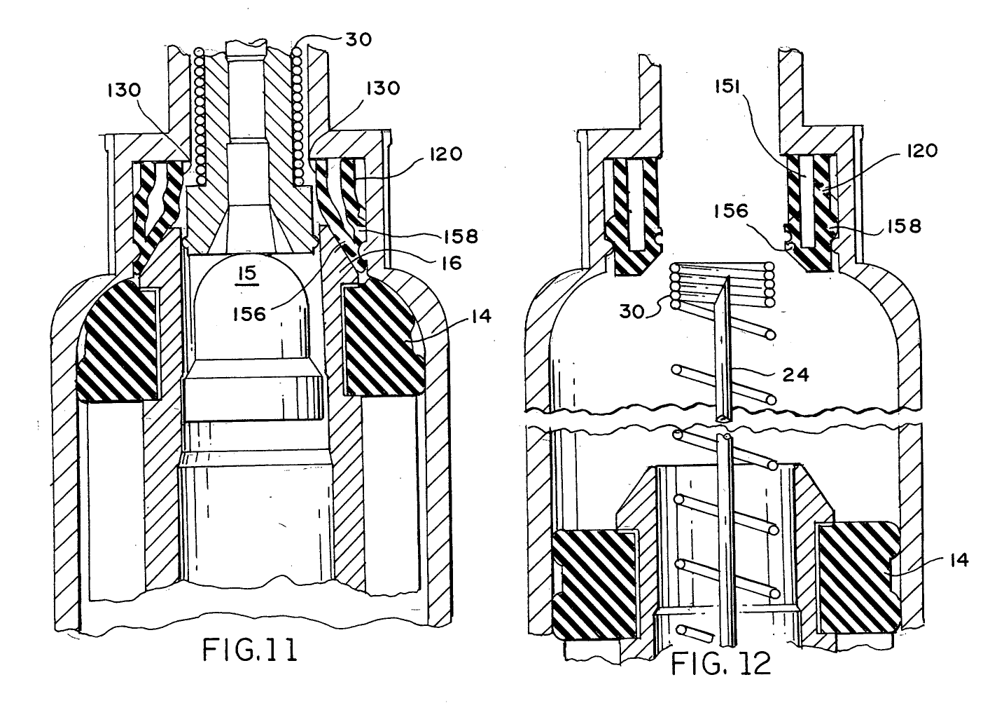

FIG. 12 is a final view of the needle fully retracted from the chuck seal alternative embodiment.

### DESCRIPTION OF THE FIRST EMBODIMENT

The environment of the subject invention is shown in FIG. 1 where the initial view leaves one with the impression that an ordinary syringe is being used. However, this ordinary syringe is provided with an extraordinary feature permitting retraction of the injection needle after the total amount of the injectant has been introduced into the patient. In use, the syringe is filled in essentially the same fashion as any syringe of the prior art.

More specifically, as shown in FIGS. 1 and 2, the syringe 10 has a barrel 11 in which a hollow piston 12 is provided. The needle assembly 19 is at one end, and a piston cap 16 at the other end which is engaged normally by the thumb of the user.

Turning now to FIG. 2, it will be seen that the syringe 10 and its barrel 11 are secured coaxially with a hollow piston 12. The hollow piston 12 has an annular piston seal 14 at one end, and a piston plug 15 interiorly of the end of the piston adjacent the piston seal 14. The piston cap 16 is at the opposite end of the piston seal 14 and is normally engaged by the thumb of the user. In order to ensure against removal, and permit simple assembly, a seal groove 18 is provided at the end of the piston 12 which receives the annular piston seal 14. The needle assembly 19 includes a needle head 21, with a needle shank 22, and a needle 24 mounted interiorly of the needle shank 22.

In accordance with the present invention, the barrel nose 25 of the barrel 11 has a hollow interior portion which not only receives the needle shank 22 and the needle head 21, but also secures the coaxially mounted spring 30 which is biased against a spring seat 31 in the barrel nose 25 and the underneath portion of the needle head 21. In this configuration, when held in place by means of the chuck seal 20, the needle is poised for retraction upon release of the chuck seal 20, but the chuck seal 20 also serves the purpose of a needle seal until such time as it is open and then permits the needle to be moved interiorly of the hollow piston 12.

As shown in FIG. 3, the contents of the barrel 11 have been injected through the needle 24, and the annular chuck seal key 26 is poised to engage the chamfered face of the chuck seal ramp 28 to thereby open the chuck seal 20 a sufficient amount to permit the force of the coaxially mounted spring 30, riding against the base of the needle head 21, to propel the needle along with the piston plug 15 interiorly throughout the length of the hollow piston until retracted to the position as shown in FIG. 4. At this time the piston cap 16 has been pressed forward to secure it in the interior groove 29 provided in the barrel head adjacent the piston cap.

Turning now to FIGS. 5 and 6, it will be seen that in FIG. 5 the chuck seal 20 overlies the needle head 21 a sufficient amount to secure the entire needle assembly against dislodgement by the force of the coaxial spring 30. Turning now to FIG. 6, it will be seen that when the annular chuck seal key 26 contacts the chuck seal ramp 28, it causes that end of the chuck seal 20 to open up to a point where the spring 30 propels the needle assembly 19 by pushing against the spring seat head 32 to dislodge the chuck seal seat 34 and open the chuck seal jaws 35 which are separated by the chuck seal slots 36. At the same time the piston cap 16 is being pushed into the interior groove 29 at the cap end of the barrel 11.

Breather space 38 is provided so that pressure does not build up at the cap end of the piston when the injectant is withdrawn from a vial. The same breather, of course, permits a flow of air into the piston barrel above the piston seal when the injection procedure is undertaken, and therefore it reduces any tendency for a negative pressure to form which could interfere with the injection process.

Specific details of the polypropylene chuck seal 20 and 120 include the following Nova Polymers, Inc., Product Specification ID: NP-NOVALAST 4001, for thermoplastic elastomer:

| | | ASTM |
|---|---|---|
| | AVERAGE VALUES | TEST METHOD |
| RESIN | | |
| Processing Methods | Injection, Extrusion, | Injection, Extrusion, |
| | Blow Molding | Blow Molding |
| Processing Temperature | 390° F./199° C. | — |
| Specific Gravity, g/cm³ | 0.953 | D-792 |
| MECHANICAL | | |
| Hardness, Shore, “A”, | 63 | D-2240 |
| 15 sec. delay | | |
| Tensile Strength, @ | 950 | D-412 |
| break, ft.lb./cm², psi | | |
| Elongation, @ break | 400% | D-412 |
| Flexural Modulus, psi | 400 | D-412 |
| Low Temperature, brittle | >−50° F. | — |
| point | | |
| Compression Set, % @ | 26.8 | — |
| 77° F., 22 hrs | | |
| Compression Set, % @ | 36.0 | — |
| 158° F., 22 hrs | | |
| Compression Set, % @ | 47.7 | — |
| 212° F., 22 hrs | | |
| Tear, die “C”, ppi | 172 | — |

### DESCRIPTION OF THE SECOND EMBODIMENT

As shown in FIG. 9, the reference numerals remain the same for parts which are common between the first embodiment and the second embodiment. As to the second embodiment, like or related parts are in the 100's series. Thus, reference numeral 120 relates to the second embodiment chuck seal 120 while the first embodiment chuck seal 20 is reference numeral 20. The second embodiment chuck seal 120 includes the chamfer 128 which is substantially the same as in the first embodiment. The portion of the second embodiment chuck seal 120 which is in the direction of the head of the needle includes an outer skirt 150, an annular groove 151, and a cylindrical inner skirt 152 which causes the two skirts to be defined by the annular groove which renders the chamfered end at the chamfered face 128 easier to pry open by the chamfer key. Continuing with the view in FIG. 8, an outer boss 155 is provided on the second embodiment chuck seal 120 to engage the barrel ring 159 to firmly position the second embodiment chuck seal 120 in a biased fashion on the interior portion of the barrel head.

Moving to FIG. 9 which is a cross-section, it will be seen that the chamfer 128 is positioned the same as in the first embodiment. Immediately behind the chamfer provision is made for a chuck seal outer boss 155 which engages the barrel ring 159. The inner core of the cylindrical inner skirt 152 is shown as reference numeral 130. The inner core diameter 130 is larger than the diameter of the head seat 156 and 158 which are respectively the outer head seat 156 and the inner head seat 158, which are both separated by means of head seat groove 160. The action of the outer and inner head seats 156, 158 occurs when the second embodiment chamfered ramp 128 is engaged by the key 26 which in essence peels the chuck jaw progressively open to permit the discharge of the needle head since the holding force of the outer head seat 156, because it is wider than the inner head seat 158, permits the needle to be released easily after the outer head seat 156 is opened.

With the second embodiment there is no requirements for slots to create the jaws, but rather it is the annular groove 151 which creates two skirts which serve to permit the chamfer face 128 to be engaged simultaneously with the release of the piston plug 15 and then the needle head 21 is propelled by the spring 30 just as in the first embodiment.

The first embodiment of the chuck seal 120 is symmetrical, and therefore easy to drop into the barrel for assembly. With the second embodiment the chuck seal 120 is secured to the needle assembly prior to insertion of the needle assembly into the barrel and thereafter the coaxial spring 30 is moved around the needle shank 22 for assembly into the barrel nose 25.

### The Method

The method of the present invention is two-fold in that it eliminates moving parts and utilizes fixed parts which are easy to orient for automated assembly. The retraction of the syringe needle assembly 19 is poised to occur substantially simultaneously with the dislodgement of the piston plug 15 and the locking securement of the piston cap 16. All of this flows from providing a chuck seal 20 which is symmetrical about its annular body. Three alternatively opposed jaws are defined by chuck seal slots 36, thereby leaving three chuck seal jaws 35 on each side of the chuck seal. The principal piston seal 14 is secured in place by the seal groove provided in the seal end of the piston. The piston plug 15 is held in place by a slight interference fit with the piston barrel end so that upon engaging the chuck seal, primarily as shown in FIG. 6, the piston plug is moved into the hollow piston barrel to be further moved by the propelling action by the spring 30 against the needle head 21. Substantially simultaneously therewith, means are provided to secure the piston cap 16 into interior groove 29 in the end of the hollow piston 12 so that the piston 12 cannot be removed after the needle has retracted to the configuration, as best shown in FIG. 4.

The method employed in the second embodiment illustrated in FIGS. 9 through 12 differs somewhat from the first embodiment since the chuck jaws of the first embodiment find their equivalent in the weakened inner and outer skirts 152 and 150 which are defined by the annular groove 151. Similarly, with the second embodiment, the portion of the chuck 120 which engages the needle head 21 is separated into an inner and outer seats 156, 158 by means of an annular ring. Thus, the method contemplates providing a strong gripping force on the inter face between the chuck seal 120 and the head of the needle adjacent the piston plug 15 to assist in reducing the amount of pressure at the end of the injection stroke which is necessary to move the piston plug 15 interiorly of the hollow piston 12 followed by the needle assembly 19 being propelled by the spring 30 into the hollow piston 12. Sufficient forces are developed so that retraction assists in the withdrawal of the needle fro the patient, particularly when the needle has been siliconized to provide for such withdrawal.

It will be understood that various changes in the details, materials and arrangements of parts, or method which have been herein described and illustrated in order to explain the nature of the invention, may be made by those skilled in the art within the principle and scope of the invention as expressed in the appended claims.

What is claimed is:

1. A syringe with retractable needle comprising, in combination,
a syringe having a barrel which is essentially cylindrical and longitudinal in configuration;
a piston which is hollow and configured for movable mounting interiorly of the barrel of the syringe;
a needle assembly;
a piston seal provided at the end of the closest to the needle assembly;
means for securing the piston seal to the syringe barrel immovably resisting any effort of the piston seal to slide longitudinally along the piston in operation;
a plug removably secured at the end of the piston closest the needle assembly which is proportioned to fit within the piston to be removably propelled when contacting the needle assembly;
said needle assembly including a spring coaxially mounted surrounding a needle, which spring is positioned to press underneath the head of the needle in biased relationship with the end of the barrel to thereby dislodge the needle assembly;
a chuck seal for securely positioning interiorly of the end of the barrel and in surrounding relationship to the needle head;
said needle head and said chuck seal being proportioned to have a resiliently secured interface attributable to the dimensioning of the chuck seal and relating it to the needle head;
said chuck seal having a chuck seal ramp at the end facing the piston plug; and
a chuck seal annular key at the seal end of the piston proportioned to engage the chuck seal ramp to release the chuck seal from the needle head;
all such elements being formed and proportioned to permit the piston, after the barrel is filled to the preselected quantity of injectant, to be moved forwardly and then substantially simultaneously at the end of the stroke of the piston dislodge the piston plug and then have the chuck seal key open the chuck seal ramp, and then spring propelledly permitting the needle assembly to be sent into the interior portion of the piston.

2. The chuck seal according to claim 1 in which the chuck seal ramp outer diameter has a ratio of substantially 3:2 to the chuck seal ramp inner diameter.

3. The chuck seal according to claim 1 in which the chuck seal length has substantially a ratio of 5:4 to the inner diameter.

4. The chuck seal according to claim 1 in which the included angle across the diameter of the chuck seal ramp is in the range of 70° to 80°.

5. In the syringe according to claim 1,
interlocking means provided at the end of the barrel for interlockingly engaging the piston cap and secure it against removal after the needle has been retracted into the syringe.

6. In the syringe according to claim 1,
said chuck seal having a plurality of jaws which are defined by peripheral slots.

7. In the chuck seal according to claim 1,
said chuck seal having ramp-like chuck seal ramps defined by an interior substantially uniform chamfer in one end of the chuck seal.

8. In the chuck seal according to claim 1,
more than one slot in the chuck seal defining jaws at both ends of the chuck seal, which chuck seal is substantially symmetrical about the plane passing through and perpendicular to its axis, whereby when the chuck seal is assembled into the syringe it can be dropped in from either directions and properly seated by a-like tool without regard for proper orientation of one end with respect to the other.

9. In the chuck seal according to claim 1,
said chuck seal being formed with an annular groove opening in the end of the chuck seal opposite the chuck seal ramp;
said groove defining an outer skirt and an inner skirt which cause the seal to be weakened at a point closely adjacent to the ramp; whereby progressive opening of the continuous jaw of the chuck seal defined at the chuck seal ramp when the chuck seal key is engaging the ramp face at the end of the stroke of the plug.

10. A method of forming a syringe with retractable needle comprising the steps of:
providing a syringe with a hollow barrel;
providing a coaxial hollow piston interiorly of the hollow barrel, a seal at one end of the hollow piston for biasing the end of the piston against the interior portion of the barrel and thereby sealing it;
adding a cap to the piston for engagement by the user and positioning a needle assembly with a coaxial spring which is secured against the needle assembly head in the end of the barrel;
the improved method of inserting a permanently fixed chuck seal at the injecting end of the barrel which chuck seal has an expandable interior in compressive relationship with the head of the needle and which is proportioned along with the positioning of the piston to substantially simultaneously dislodge the piston end plug at the same time the chuck seal is engaged by an annular key at the end of the piston which opens the chuck seal and permits the retractable needle to be propelled into place interiorly of the hollow piston.

11. In a syringe including a barrel, a hollow piston mounted coaxially in the barrel, a piston seal, a piston plug, and a piston cap with the piston seal located in a groove at the opposite end of the piston from the piston cap, and the piston plug positioned at the end of the piston adjacent the piston seal and removably secured therein for shifting interiorly of the barrel, said piston being secured coaxially and interiorly of said barrel, a chuck seal key at the end of the piston closest to the needle head, a barrel nose at the end of the barrel opposite the cap, a needle assembly secured interiorly of the barrel nose, said needle assembly including a shank, needle head at one end, a needle secured within the needle shank with its discharged portion removed from a needle head, a spring comprehensibly biasing the needle to be moved into the hollow piston, the improvement comprising:
an annular elastomeric symmetrical chuck seal having an outer diameter, an inner diameter, a body, and a chuck seal ramp at one end commencing at the inner diameter and progressing angularly radially outwardly from the inner diameter;
weakening means in said chuck seal to permit bending the seal when the ramp is engaged by the chuck key in one of said ramps spaced to define chuck seal jaws therebetween;
said chuck seal having a diameter to fit within a chuck seal groove at the end of the barrel nose to bridge and seal the space between the interior portion of the barrel nose and the needle head which, upon activation by spreading the jaws releases the needle head for yieldable propulsion into the interior portion of the.

12. In the syringe according to claim 11,
said chuck seal being provided with equi-spaced slots at both ends defining jaws at both ends.

13. In the syringe according to claim 11,
said seal at the end of said being secured in a groove to thereby ensure the immovability of the seal longitudinally along the piston so that when the chuck seal key is positioned interiorly of the jaws of the chuck seal a release of the needle head will be initiated at substantially the same time and positioning of the chuck seal key against the jaws of the chuck seal.

14. The chuck seal according to claim 11 in which the jaw diameter has a ratio of substantially 3:2 to the inner diameter.

15. The chuck seal according to claim 11 in which the length has substantially a ratio of 5:4 to the inner diameter.

16. The chuck seal according to claim 11 in which the included angle across the diameter of the ramp is in the range of 70° to 80°.

17. In the chuck seal according to claim 12,
said slots being spaced at radial equal distances, and spaced at each end and staggered positioned to thereby permit the chuck seal to be inserted in either direction in a seat provided in the barrel nose.

18. An elastomeric chuck seal for use in a syringe having a barrel and a barrel nose to receive a needle assembly and a piston in said barrel for removing and retaining a needle assembly having a head which is in spring loaded relationship to the syringe, said chuck seal comprising:
an annular elastomeric member, cylindrical in nature, said member having an inner and an outer cylindrical surfaces;
a frusto conical ramp at one opening to the inner diameter;
said ramp inclining radially outwardly defining a frusto conical ramp surface;
a cylindrical annular undercut in the end of the seal opposite the ramp defining two coaxial skirts;
the chuck seal having a body formed and proportioned to fit within a chuck seal chamber at the nose portion of the barrel in surrounding and seating relationship to the needle assembly and having sufficient interference with the needle head to hold the same in place against a resilient load applied to the needle head and coaxially with the needle in the direction of the piston and said chuck seal is immovably secured coaxially along the needle head to thereby continually seal the needle during the entire period of injection and retraction of the needle head into the syringe body.

19. The chuck seal according to claim 18 in which the outer diameter bears a ratio to the inner diameter of substantially 2:1.

20. The chuck seal according to claim 18 in which the jaw diameter has a ratio of substantially 3:2 to the inner diameter.

21. The chuck seal according to claim 18 in which the length has substantially a ratio of 5:4 to the inner diameter.

22. The chuck seal according to claim 18 in which the included angle across the diameter of the ramp is in the range of 70° to 80°.

23. The chuck seal according to claim 18 which is formed from a polymer having the following specifications:
a specific gravity of 0.953 and an elongation at break of at least 400%.

24. An elastomeric chuck seal in a syringe for first retaining and thereafter removing a needle assembly having a head which is in spring loaded relationship to the syringe, said chuck seal comprising:
an annular elastomeric member, cylindrical in nature, said cylinder having an inner an an outer cylindrical surfaces;
frusto conical ramp at one opening to the inner diameter;
said ramp inclining radially outwardly defining a frusto conical ramp surface;
the body of the chuck seal extending away from the chuck seal ramp having an annular groove to thereby define an outer skirt and an inner skirt in which the outer skirt particularly collapses in one inner skirt when the ramp is engaged and opened;
the chuck seal having a body formed and proportioned to fit within a chuck seal chamber at the nose portion of the barrel in surrounding and needle head to hold the same in place against a resilient load applied to the needle head and coaxially with the needle in the direction of the piston and said chuck seal is immovably secured coaxially along the needle head to thereby continually seal the needle during the entire period of injection and retraction of the needle head into the syringe body.

25. The chuck seal according to claim 24 in which the outer diameter bears a ratio to the inner diameter of substantially 2:1.

26. The chuck seal according to claim 24 in which the jaw diameter has a ratio of substantially 3:2 to the inner diameter.

27. The chuck seal according to claim 24 in which the length has substantially a ratio of 5:4 to the inner diameter.

28. The chuck seal according to claim 24 in which the included angle across the diameter of the ramp is in the range of 70° to 80°.

29. The chuck seal according to claim 24 which is formed from a polymer having the following specifications:
a specific gravity of 0.953 and an elongation at break of at least 400%.
